SEQUENCE LISTING (10 sequences)

SEQ ID NO: 1 - DNA, 102 bp, synthetic construct
  source 1..102: mol_type = other DNA
gcccagcccctgcccgactgctgcaggcagaagacctgcagctgcaggctgtacgagctgctgcacggcgccggcaaccacgccgccggcatcctgaccctg

SEQ ID NO: 2 - DNA, 84 bp, synthetic construct
  source 1..84: mol_type = other DNA
aggaggagcggcccccccggcctgcagggcaggctgcagaggctgctgcaggccagcggcaaccacgccgccggcatcctgacc

SEQ ID NO: 3 - DNA, 29 bp, synthetic construct
  source 1..29: mol_type = other DNA
gggaatcacatcagtaactgagtgacttc

SEQ ID NO: 4 - DNA, 396 bp, synthetic construct
  source 1..396: mol_type = other DNA
atggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaa

SEQ ID NO: 5 - DNA, 33 bp, synthetic construct
  source 1..33: mol_type = other DNA
tatggccgcaaaaaacgccgccagcgccgccgc

SEQ ID NO: 6 - DNA, 517 bp, synthetic construct
  source 1..517: mol_type = other DNA
ggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatg

SEQ ID NO: 7 - DNA, 99 bp, synthetic construct
  source 1..99: mol_type = other DNA
atgaacctgcccagcaccaaggtgagctgggccgccgtgaccctgctgctgctgctgctgctgctgccccccgccctgctgagcagcggcgccgccgcc

SEQ ID NO: 8 - DNA, 884 bp, synthetic construct
  source 1..884: mol_type = other DNA
gggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggcctacccctacgacgtgcccgactacgccctggccaccacccatatgggatccgaattcatgaacctgcccagcaccaaggtgagctgggccgccgtgaccctgctgctgctgctgctgctgctgccccccgccctgctgagcagcggcgccgccgccgcccagcccctgcccgactgctgcaggcagaagacctgcagctgcaggctgtacgagctgctgcacggcgccggcaaccacgccgccggcatcctgaccctgctcgagtgagatctgactgaaaaaaaaaaaaaaaaaaaaaaaaaaaaaagtttaaacactagtccgctgagcaataa

SEQ ID NO: 9 - DNA, 866 bp, synthetic construct
  source 1..866: mol_type = other DNA
gggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggcctacccctacgacgtgcccgactacgccctggccaccacccatatgggatccgaattcatgaacctgcccagcaccaaggtgagctgggccgccgtgaccctgctgctgctgctgctgctgctgccccccgccctgctgagcagcggcgccgccgccaggaggagcggcccccccggcctgcagggcaggctgcagaggctgctgcaggccagcggcaaccacgccgccggcatcctgaccctcgagtgagatctgactgaaaaaaaaaaaaaaaaaaaaaaaaaaaaaagtttaaacactagtccgctgagcaataa

SEQ ID NO: 10 - DNA, 540 bp, synthetic construct
  source 1..540: mol_type = other DNA
ggaattgtgagcggataacaattcccctgtagaaataattttgtttaactttaataaggagatataccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatcaggatcaggatatggccgcaaaaaacgccgccagcgccgccgtgacctaggctgctgccaccgctgagcaataa

(19) United States
(12) Patent Application Publication
(10) Pub. No.: US 2024/0398722 A1
(43) Pub. Date: Dec. 5, 2024

(54) COMPOSITIONS AND METHODS FOR PRODUCING OREXIN NEUROPEPTIDE USING NANOCAPSULE-BASED DRUG DELIVERY SYSTEM

(71) Applicant: VL Biotech Inc., New Taipei (TW)

(72) Inventors: Hsin-Yi LIN, New Taipei (TW); Stanley KUNG, New Taipei (TW)

(21) Appl. No.: 18/328,561

(22) Filed: Jun. 2, 2023

(51) Int. Cl. A61K 9/51; C07K 14/005; C07K 14/47; C12N 15/85

(52) U.S. Cl. CPC A61K 9/5184; C07K 14/005; C07K 14/4705; C12N 15/85; A61K 48/00; C12N 2840/203

(57) ABSTRACT

Disclosed herein are compositions and methods with enhanced capability to produce orexin neuropeptide. The compositions are loaded in a nanocarrier for drug delivery. The composition comprises a first vector and a second vector. The first vector encodes mRNAs encoding a peptide with orexin activity is labeled with one or more capsid protein tags. The second vector encodes one or more capsid proteins and BBB-penetrating peptides. The one or more capsid proteins bind to the one or more capsid protein tags on the one or more mRNA.

### REFERENCE TO AN ELECTRONIC SEQUENCE LISTING

The content of the electronic sequence listing (23HPUS0014_sequencing list.xml; Size: 12KB; and Date of Creation: May 29, 2023) is herein incorporated by reference in its entirety.

### FIELD OF THE INVENTION

The present invention relates to compositions and methods of use, in particular to therapeutic compositions with enhanced capability to produce orexin neuropeptide in a human or animal subject, which are delivered by using nanocapsule-based drug delivery system. Compositions and methods of the present invention are particularly applicable to a range of disorders, including narcolepsy, Alzheimer's disease (AD), Parkinson's disease (PD), and frontotemporal dementia.

### BACKGROUND OF THE INVENTION

Orexin-A and orexin-B, also known as hypocretin, are neuropeptides that regulate our wakefulness and appetite. People who cannot normally regulate orexin-A and/or orexin-B may have the symptoms of narcolepsy include excessive daytime sleepiness (EDS), sudden loss of muscle tone known as cataplexy, sleep paralysis, and aberrant rapid eye movement (REM) sleep. Also, numerous studies have reported that patients with neurodegenerative diseases such as Alzheimer's disease (AD), Parkinson's disease (PD), frontotemporal dementia, etc., have decreased numbers of orexigenic neurons and lowered orexin in plasma or cerebral spinal fluid (CSF).

Recently, researchers discovered that the motor and cognitive performance in the AD/PD mouse model are improved after using orexin replacement therapy. However, delivering the synthesized orexin to the brain cells finds it very challenging, and has become the hurdle of this approach.

### SUMMARY OF THE INVENTION

The present disclosure provides a composition for producing orexin neuropeptide. The composition comprises a first vector and a second vector. The first vector comprises the nucleotide sequences as denoted by SEQ ID NO: 1 and SEQ ID NO: 2, and a capsid protein recognition sequence. The first vector encodes mRNAs encoding a peptide with orexin activity, and the mRNAs bind to one or more capsid protein tags. The second vector comprises one or more nucleotide sequences comprises the nucleotide sequence as denoted by SEQ ID NO: 4 and SEQ ID NO: 5. The second vector encodes one or more capsid proteins and BBB-penetrating peptides. The one or more capsid proteins are capable of self-assembling into one or more virus-like particles that encapsulate the mRNAs such that the composition is fabricated as nanocapsule.

The present disclosure also provides a composition for producing orexin neuropeptide. The composition comprises a first vector and a second vector. The first vector comprises the nucleotide sequences as denoted by SEQ ID NO: 8 and SEQ ID NO: 9. The second vector comprises the nucleotide sequence as denoted by SEQ ID NO: 10.The nucleotide sequence of SEQ ID NO: 10 encodes one or more capsid proteins and BBB-penetrating peptides, and the one or more capsid proteins are capable of self-assembling into one or more virus-like particles that encapsulate mRNAs encoded by the nucleotide sequence of SEQ ID NO: 8 and SEQ ID NO: 9 such that the composition is fabricated as nanocapsule.

The present disclosure further provides a method for producing orexin neuropeptide in a subject. The method includes administering a composition comprising one or more nanocapsules and/or microbial cells containing the one or more nanocapsules to the subject. The one or more nanocapsules are formed from a first vector and a second vector. The first vector encodes mRNAs encoding a peptide with orexin activity. The orexin activity may be orexin-A activity, orexin-B activity or the combination thereof. The mRNAs are labeled with one or more capsid protein tags. The second vector comprises one or more nucleotide sequences encoding one or more capsid proteins and BBB-penetrating peptides. The one or more capsid proteins are capable of self-assembling into one or more virus-like particles that encapsulate the mRNAs such that the composition is fabricated as the one or more nanocapsules or/and the microbial cells containing the one or more nanocapsules.

### BRIEF DESCRIPTION OF THE DRAWINGS

FIG. 1 illustrates a functional orexin assay indicating the calcium flux when the orexin peptide activates the orexigenic receptor of cell.

FIG. 2 illustrates immunohistochemistry (IHC) staining of the brain tissue of the test hamster, demonstrating the orexin is produced in the test hamster after administration of the Composition 1.

### DETAILED DESCRIPTION OF THE PREFERRED EMBODIMENTS

As used herein, the term “vector” used in reference to delivery of components refers to a nucleic acid capable of transporting between different genetic environments another nucleic acid to which it has been operatively linked. The term “express” in the context of the disclosure refers to transcription and/or translation of a specific nucleotide sequence driven by its promoter.

The disclosure relates to one or more nucleotide sequences encoding a peptide with orexin activity, which particularly can be assembled into nanocapsule in bacteria (such as probiotics), for increasing orexin neuropeptide in a subject. Thus, the one or more nucleotide sequences can be incorporated into pharmaceutical and/or therapeutic compositions suitable for administration. Unless specified otherwise, the term “orexin activity” used in this disclosure may be orexin-A activity, orexin-B activity or the combination thereof.

The present disclosure discloses a first vector and a second vector, the first vector encodes one or more messenger ribonucleic acids (messenger RNAs, mRNAs) encoding a peptide with orexin activity. The mRNAs are further labeled with one or more capsid protein tags. The second vector encodes one or more capsid proteins (CPs) and BBB-penetrating (blood-brain barrier-penetrating) peptides, which may be self-assembled to form one or more hollow virus-like particles (virus-like particles, VLPs), then bind to and provide domain specificity to the one or more capsid protein tags on the mRNAs, such that the one or more mRNAs are capable of being encapsulated in the one or more VLPs. Thus, the mRNAs encoding the peptide with orexin activity are carried in nanocarrier and will be delivered via nanocapsules after intramuscular injections and inhalation.

In some aspects, the pharmaceutical and/or therapeutic compositions comprise bio-products or compounds transcribed or translated from the first vector and the second vector described in the present disclosure.

The pharmaceutical and/or therapeutic composition may be used to produce orexin neuropeptide. Therefore, in one aspect, it is contemplated that the compositions and methods of the disclosure are used to treat and/or prevent neurodegenerative diseases, such as narcolepsy, Alzheimer's disease (AD), Parkinson's disease (PD), and frontotemporal dementia.

In one aspect, the disclosure relates to one or more nanocapsule containing bacteria, which is assembled from one or more bio-products transcribed or translated from the first vector and the second vector. The bio-products may be either the mRNAs or the capsid proteins.

In another aspect, the disclosure relates to one or more nanocapsule assembled from one or more bio-products transcribed or translated from the first vector and the second vector. The bio-products may be either the mRNAs or the capsid proteins.

In yet another aspect, the disclosure relates to one or more VLPs assembled from one or more bio-products transcribed or translated from the first vector and the second vector. The bio-products may be either the mRNAs or the capsid proteins.

In further aspect, the disclosure provides a use of the pharmaceutical and/or therapeutic composition of the disclosure in a preparation of a medicament for a treatment of neurodegenerative diseases, such as narcolepsy, Alzheimer's disease (AD), Parkinson's disease (PD), and frontotemporal dementia.

In another aspect, the disclosure provides a method for treating and/or preventing neurodegenerative diseases, such as narcolepsy, Alzheimer's disease (AD), Parkinson's disease (PD), and frontotemporal dementia, which comprises administering the pharmaceutical and/or therapeutic composition of the disclosure or the preparation thereof.

In one embodiment, the first vector comprises the nucleotide sequences as denoted by SEQ ID NO: 1 and SEQ ID NO: 2, and a capsid protein recognition sequence. The first vector is transcribed to form mRNAs with the one or more capsid protein tags, which encodes a peptide with orexin activity, and the mRNAs can be used as a translation template in a cell of a human or animal subject to produce a peptide with orexin activity in the human or animal subject's body, to endogenously produce orexin-A and orexin-B in vivo. In addition, the one or more capsid protein tags can bind to a specific region of one or more capsid proteins because of a secondary structure formed by the capsid protein recognition sequence. In one embodiment, the capsid protein recognition sequence comprises the nucleotide sequence as denoted by SEQ ID NO: 3, and the one or more capsid protein tags formed by the capsid protein recognition sequence has a hairpin structure.

In the present disclosure, one specific nucleotide sequence may be included in a single vector, for example, the nucleotide sequence of SEQ ID NO: 1 and the nucleotide sequence of SEQ ID NO: 2 may be separately included in different vectors. Alternatively, more than one specific nucleotide sequences may be included in a single vector, for example, the nucleotide sequence of SEQ ID NO: 1 and the nucleotide sequence of SEQ ID NO: 2 may be included in a same vector.

Further, the second vector comprises one or more nucleotide sequences encoding the one or more capsid proteins and BBB- penetrating peptides. In one embodiment, the one or more capsid proteins are shell proteins derived from bacteriophages, such as AP205 phage, Qbeta phage, MS2 phage, P22 phage and the like. In another embodiment, the second vector comprises the nucleotide sequence as denoted by SEQ ID NO: 4, and the one or more capsid proteins encoded by the nucleotide sequence of SEQ ID NO: 4 can be bound with the one or more capsid protein tags formed by the nucleotide sequence of SEQ ID NO: 3. Further, the second vector comprises the nucleotide sequence as denoted by SEQ ID NO: 5,which promotes the nanocapsule enters the blood-brain barrier of the subject.

The first vector of the composition expresses the mRNAs encoding the peptide with orexin activity, and the second vector of the composition expresses the one or more capsid proteins and BBB-penetrating peptides. The one or more capsid protein tags on the mRNAs facilitate the combination of the capsid proteins and the mRNAs. Consequently, the one or more capsid proteins are self-assembled into the one or more virus-like particles which can be utilized to deliver the mRNAs encoding the peptide with orexin activity in the form of nanocapsules.

The first vector may further comprise an internal ribosome entry site (IRES) containing the nucleotide sequence as denoted by SEQ ID NO: 6. The internal ribosome entry site is placed between the capsid protein recognition sequence and the nucleotide sequences of SEQ ID NO: 1 and SEQ ID NO: 2. Moreover, the first vector may further comprise the nucleotide sequence as denoted by SEQ ID NO: 7, which encodes a secretion signal for promoting the synthesis of the peptide with orexin activity. The first vector may include a promoter and a terminator for RNA polymerase to perform transcription.

In another embodiment, the composition of the disclosure comprises a first vector and a second vector. The first vector comprises the nucleotide sequence as denoted by SEQ ID NO: 8 and SEQ ID NO: 9 encoding one or more capsid protein tags and mRNAs which encodes a peptide with orexin activity. The second vector comprises the nucleotide sequence as denoted by SEQ ID NO: 10encoding one or more capsid proteins and BBB-penetrating peptides. The one or more capsid proteins encoded by the nucleotide sequence of SEQ ID NO: 10 can be combined with the one or more capsid protein tags formed by the nucleotide sequence of SEQ ID NO: 8 and SEQ ID NO: 9.

The disclosure further provides a nanocapsule-based drug delivery system for delivering mRNA into the body of the human or animal subject to produce orexin neuropeptide of the human or animal subject. In one embodiment, the nanocapsule-based drug delivery system is achieved by various administrations, which are used to enable delivery of the compositions to the desired site of biological action. These administrations include, but are not limited to oral routes, intraduodenal routes, parenteral injection (including intravenous, subcutaneous, intraperitoneal, intramuscular, intravascular or infusion), topical and rectal administration.

In one embodiment, the first vector and the second vector are transformed into a transformed cell, and the genetic information of the first vector and the second vector are expressed through the physiological mechanism of the transformed cell. In an example, the transformed cells are bacterial cells. In the process of the transformation (or the bacterial cells are produced), the first vector and the second vector are either transcribed into mRNAs or translated into capsid proteins; the bio-products are then self-assembled into the VLPs, encapsulated in the bacterial cells. In an alternative example, the transformed cells are probiotics. In the process of the transformation (or the probiotics are produced), the first vector and the second vector are either transcribed into mRNAs or translated into capsid proteins; the bio-products are then self-assembled into the VLPs, encapsulated in the probiotics.

Specifically, the first vector is expressed by the transformed cell to produce the mRNAs with the one or more capsid protein tags, and the mRNAs encode a peptide with orexin activity in the body of the human or animal subject. The second vector is expressed by the transformed cell to form one or more capsid proteins and BBB-penetrating peptides. The products of the first vector and the second vector can be self-assembled into nanocapsule in the transformed cell by binding of the one or more capsid protein tags to the one or more capsid proteins.

The composition of the disclosure can promote the body of the human or animal subject to produce the peptides with orexin-A and orexin-B activity, which are more similar to endogenous orexin-A and orexin-B than artificially synthesized orexin-A and orexin-B derivatives. The orexin-A and orexin-B produced in vivo are neuropeptides that regulate wakefulness and appetite, so as to enable therapeutic effects by producing orexin neuropeptide.

In one embodiment, the transformed cells are bacterial cells, which can be easily obtained and cultured from bacterial cultures. The bacterial cells may be Escherichia coli.

The following examples are disclosed to demonstrate that the composition of the disclosure is able to endogenously produce orexin-A and orexin-B in vivo, causing a production of orexin neuropeptide. The following examples are only for the purpose of illustration, and the scope of the disclosure is not limited by the examples. Those skilled in the art can utilize the disclosure and teachings of the disclosure to create other embodiments, aspects and variations without undue experimentation.

### Example 1

The Composition 1 is prepared by transforming the first vector and the second vector into Escherichia coli cells. The first vector adopts pT7CFE1-NHA vector and contains the nucleotide sequence of SEQ ID NO: 8 and SEQ ID NO: 9. The second vector adopts pCDF-1b vector and contains the nucleotide sequence of SEQ ID NO: 10. Then the Composition 1 is administered to eukaryotic host cells, HEK293 cells, by adding into the culture medium of HEK293 cells and cultured overnight.

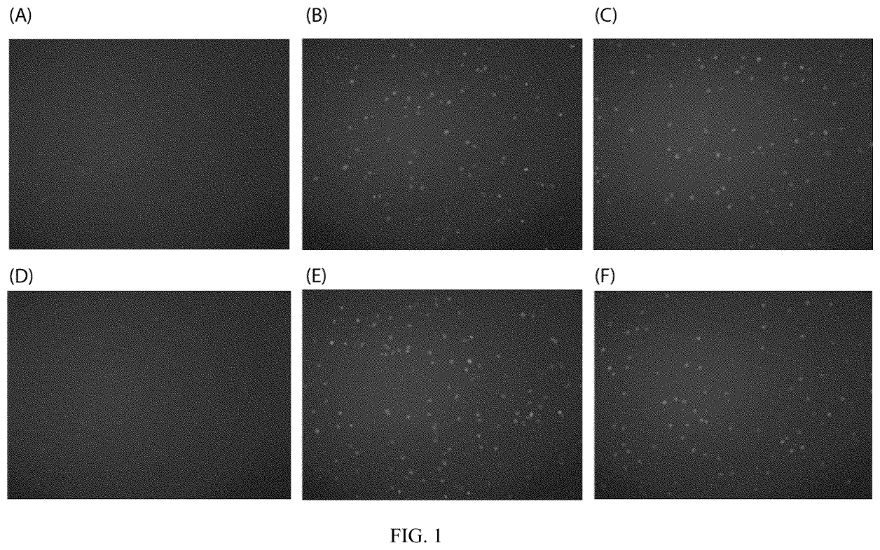

The overnight cultured medium was then added to the Chem-1 cell for functional orexin assay. If the mRNA nano-capsule enables HEK293 to produce orexin, the HEK293-produced orexin from the culture medium will trigger calcium flux by binding to the orexigenic receptor of the Chem-1, and the calcium flux can be detected by the fluorescent signal. In FIG. 1, figures (A) and (D) are negative controls, figure (B) is the fluorescent image of the Chem-1 cell added with the cultured medium including HEK293-produced orexin-A via orexin-A mRNA nanocapsule, figure (E) is the fluorescent image of the Chem-1 cell added with the cultured medium including HEK293-produced orexin-B via orexin-B mRNA nanocapsule, (C) and (F) are positive control by adding commercially available orexin-A and orexin B. The result indicates that the orexin-A/orexin-B mRNA nanocapsule confer the HEK293 capability of orexin production.

### Example 2

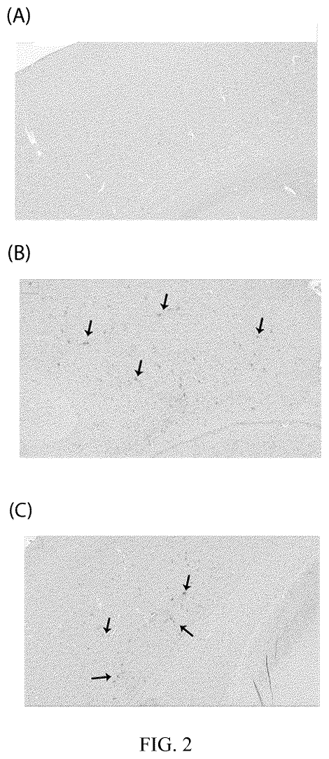

Hamster was used to demonstrate the effect of producing the orexin neuropeptide in vivo. Composition 1 is delivered to the test hamster by intramuscular injections or inhalation, and the primary cerebral cortex's immunohistochemistry (IHC) staining indicates whether the orexin is produced. In FIG. 2, figure (A) is the control group, figure (B) is the image of the brain tissue stained by the orexin-A antibody after the test hamster is given the Composition 1 through intramuscular injections, figure (C) is the image of the brain tissue stained by the orexin-A antibody after the test hamster is given the Composition 1 through inhalation. The black arrow indicates the in-house orexin-A stained by the antibody. It can be clearly seen from FIG. 2 that orexin-A is generated in the intramuscular injections and inhalation treatment group.

Further, a real-time PCR is applied to detect the level of the orexin mRNA in the tissue. In the results, the level of the orexin mRNA of the intramuscular injections group is 70 times higher than the control, and the level of the orexin mRNA of the inhalation group is 14 times higher than the control. Consequently, the results indicate that the Composition 1 can penetrate the BBB and reach the brain's cortex, olfactory bulb, and hippocampus areas, other than the hypothalamus, to produce the orexin neuropeptide, hence being expected to be useful for treating disorders of narcolepsy.

What is claimed is:

1. A composition for producing orexin neuropeptide, comprising:
a first vector, comprising nucleotide sequences as denoted by SEQ ID NO: 1 and SEQ ID NO: 2, and a capsid protein recognition sequence, wherein the first vector encodes mRNAs encoding a peptide with orexin activity, and the mRNAs are labeled with one or more capsid protein tags; and
a second vector, comprising one or more nucleotide sequences encoding one or more capsid proteins and BBB-penetrating peptides, wherein the one or more capsid proteins are capable of self-assembling into one or more virus-like particles that encapsulate the mRNAs such that the composition is fabricated as nanocapsule.

2. The composition according to claim 1, wherein the capsid protein recognition sequence in the first vector comprises a nucleotide sequence as denoted by SEQ ID NO: 3, and the second vector comprises the one or more nucleotide sequences as denoted by SEQ ID NO: 4 and SEQ ID NO: 5.

3. The composition according to claim 1, wherein the first vector further comprises an internal ribosome entry site (IRES) between the capsid protein recognition sequence and the nucleotide sequences of SEQ ID NO: 1 and SEQ ID NO: 2.

4. A composition for producing orexin neuropeptide, comprising:
a first vector, comprising nucleotide sequences as denoted by SEQ ID NO: 8 and SEQ ID NO: 9; and
a second vector, comprising one or more nucleotide sequences as denoted by SEQ ID NO: 10;
wherein the one or more nucleotide sequences of SEQ ID NO: 10 encode one or more capsid proteins and BBB-penetrating peptides, and the one or more capsid proteins are capable of self-assembling into one or more virus-like particles that encapsulate mRNAs encoded by the nucleotide sequences of SEQ ID NO: 8 and SEQ ID NO: 9 such that the composition is fabricated as nanocapsule.

5. A method for producing orexin neuropeptide in a subject, comprising:
administering a composition comprising one or more nanocapsules and/or microbial cells containing the one or more nanocapsules to the subject, wherein the one or more nanocapsules are formed from a first vector and a second vector, the first vector encoding mRNAs, wherein the mRNAs encode a peptide with orexin activity, and the mRNAs are labeled with one or more capsid protein tags, the second vector comprising one or more nucleotide sequences encoding one or more capsid proteins and BBB-penetrating peptides, wherein the one or more capsid proteins are capable of self-assembling into one or more virus-like particles that encapsulate the mRNAs such that the composition is fabricated as the one or more nanocapsules and/or the microbial cells containing the one or more nanocapsules.

6. The method according to claim 5, wherein the first vector comprises nucleotide sequences as denoted by SEQ ID NO: 1 and SEQ ID NO: 2, and a capsid protein recognition sequence.

7. The method according to claim 6, wherein the capsid protein recognition sequence in the first vector comprises a nucleotide sequence as denoted by SEQ ID NO: 3, and the second vector comprises the one or more nucleotide sequences as denoted by SEQ ID NO: 4 and SEQ ID NO: 5.

8. The method according to claim 6, wherein the first vector further comprises an internal ribosome entry site (IRES) between the capsid protein recognition sequence and the nucleotide sequences of SEQ ID NO: 1 and SEQ ID NO: 2.

9. The method according to claim 5, wherein the first vector comprises nucleotide sequences as denoted by SEQ ID NO: 8 and SEQ ID NO: 9, and the second vector comprises the one or more nucleotide sequences as denoted by SEQ ID NO: 10.
